(19) United States
(12) Patent Application Publication
(10) Pub. No.: US 2018/0014789 A1
(43) Pub. Date: Jan. 18, 2018

(54) HEART SOUND DENOISING APPARATUS, METHOD, AND PROGRAM

(71) Applicants: KYUSHU INSTITUTE OF TECHNOLOGY, Fukuoka (JP); AI TECHNOLOGY, INC., Fukuoka (JP)

(72) Inventor: Yasushi Sato, Fukuoka (JP)

(21) Appl. No.: 15/715,991

(22) Filed: Sep. 26, 2017

Related U.S. Application Data
(63) Continuation of application No. PCT/JP2016/057660, filed on Mar. 10, 2016.

(30) Foreign Application Priority Data
Mar. 26, 2015 (JP) 2015-063981

(51) Int. Cl. A61B 5/00; A61B 5/024

(52) U.S. Cl. CPC A61B 5/7203; A61B 5/725; A61B 5/024; A61B 5/7235

(57) ABSTRACT

Provided is a heart sound denoising apparatus that includes: a heart sound signal acquisition unit that acquires a heart sound signal collected by a sound collector; a pulsation signal acquisition unit that acquires a pulsation signal of a living body; a peak interval acquisition unit that acquires a peak interval of the acquired pulsation signal; a first resampling unit that resamples the heart sound signal corresponding to the acquired peak interval, to a predetermined number of samples, with respect to the heart sound signal detected at a same time as the acquired pulsation signal; a noise removal unit that removes a noise component from the resampled heart sound signal; and a second resampling unit that resamples the heart sound signal resampled by the first resampling unit and with the noise removed, to a number of samples of the peak interval.

### CROSS REFERENCE TO RELATED APPLICATIONS

This application is a continuation of International Patent Application No. PCT/JP2016/057660, filed on Mar. 10, 2016, the disclosure of which including the specification, drawings and abstract is incorporated herein by reference in its entirety. International Patent Application No. PCT/JP2016/057660 is entitled to and claims the benefit of Japanese Patent Application No. 2015-063981, filed on Mar. 26, 2015, the disclosure of which including the specification, drawings and abstract is incorporated herein by reference in its entirety.

### BACKGROUND

### Technological Field

The present invention relates to a heart sound denoising apparatus, method, and program for removing a noise included in a heart sound.

### Description of Related Art

As a technique for measuring the most effective optimum exercise intensity for a person to be measured, a technique disclosed in Japanese Unexamined Patent Publication No. 2009-297106 (hereinafter referred to as Patent Document 1) is known, for example. The technique disclosed in Patent Document 1 includes: a heart sound collecting unit 2 including a heart sound microphone 21 for collecting a heart sound and an A/D converting unit 23 for converting a heart sound signal into heart sound data; an electrocardiac collecting unit 3 including a measuring electrode 31 for measuring an electrocardiac signal and an AD converting unit 33 for converting an electrocardiac signal from the measuring electrode 31 into electrocardiac data; a reference timing detecting unit 43 for detecting an R wave from the electrocardiac data; a gate signal generating unit 44 for outputting a gate signal indicating a predetermined period from the timing of the R wave detected by the reference timing detecting unit 43 up until the second heart sound corresponding to the R wave; and a first heart sound detecting unit 45 for extracting a peak waveform as a first heart sound amplitude data from the heart sound data while the gate signal is output.

Further, as a technique for removing a noise from a signal, a technique disclosed in International Publication No. 2014/084162 (hereinafter referred to as Patent Document 2) is known, for example. The technique disclosed in Patent Document 2 includes: a peak detection unit 21 that detects a waveform peak in an input signal to be processed; a first resampling unit 24 that resamples the number of detected samples between each pair of the detected peaks to the number of reference samples; an orthogonal transformation unit 25 that performs orthogonal transformation on the resampled signal to be processed; a filtering unit 26 that extracts, from frequency data after the orthogonal transformation, at least a fundamental frequency component which is a frequency component of a fundamental wave and a harmonic component which is a frequency component of a harmonic wave; an inverse orthogonal transformation unit 27 that performs inverse orthogonal transformation on the filtered frequency data; and a second resampling unit 28 that resampies the signal to be processed which is obtained after the inverse orthogonal transformation to the number of detected samples for each pair of the peaks detected by the peak detection unit 21.

In the technique disclosed in Patent Document 1, a peak waveform is extracted from the heart sound data in a predetermined period from the timing of an R wave up until the second heart sound corresponding to the R wave to make it the first heart sound amplitude data. However, the heart sound data particularly during exercise contains many noise, and there is a problem that it is extremely difficult to extract a proper peak waveform.

In the technique disclosed in Patent Document 2, unwanted noise is reliably removed by resampling a peak interval of a signal to a predetermined number of samples and extracting only a fundamental frequency component and a frequency component of a harmonic wave. However, heart sound data particularly during exercise contains many noises, and there is a problem that it is impossible to detect the peak interval.

### SUMMARY

The present invention provides a heart sound denoising apparatus, method, and program that can detect an accurate heart sound signal by resampling a heart sound signal using a peak interval of an electrocardiac signal to remove a noise.

To achieve at least one of the above-mentioned objects, according to an aspect of the present invention, a heart sound denoising apparatus reflecting one aspect of the present invention is provided with: a heart sound signal acquisition unit that acquires a heart sound signal collected by a sound collector; a pulsation signal acquisition unit that acquires a pulsation signal of a living body; a peak interval acquisition unit that acquires a peak interval of the acquired pulsation signal; a first resampling unit that resamples the heart sound signal corresponding to the acquired peak interval, to a predetermined number of samples, with respect to the heart sound signal detected at a same time as the acquired pulsation signal; a noise removal unit that removes a noise component from the resampled heart sound signal; and a second resampling unit that resamples the heart sound signal resampled by the first resampling unit and with the noise removed, to a number of samples of the peak interval.

As described above, in the heart sound denoising apparatus reflecting one aspect of the present invention, a peak interval of a pulsation signal is acquired; with respect to a heart sound signal detected at a same time as the pulsation signal, the heart sound signal corresponding to the acquired peak interval is resampled to a predetermined number of samples; a noise component is removed from the resampled heart sound signal; and the resampled heart sound signal is again resampled to an original number of samples, so that it is possible to extract only a heart sound component according to the peak interval acquired from the pulsation signal, from the heart sound signal which is made up of a majority of external noise components (such as a worn sound of clothes during exercise, an environmental sound, and a wind sound, for example) and to remove most of the noise.

In the heart sound denoising apparatus reflecting one aspect of the present invention, the pulsation signal is an electrocardiac signal indicating an electrical change associated with a pulsation, or a pulse wave signal indicating a change in a flow of blood associated with the pulsation.

As described above, in the heart sound denoising apparatus reflecting one aspect of the present invention, it is possible to detect an accurate heart sound signal from which the noise has been removed, since a signal as a basis for removing the noise of the heart sound signal can be acquired more accurately even during exercise and is an electrocardiac signal or pulsation signal which is synchronized with the heart sound signal.

In the heart sound denoising apparatus reflecting one aspect of the present invention, the noise removal unit is provided with: an orthogonal transformation unit that orthogonally transforms the heart sound signal resampled by the first resampling unit; a filtering unit that extracts at least a fundamental frequency component and a harmonic component from frequency data after the orthogonal transformation, the fundamental frequency component being a frequency component of a fundamental wave, the harmonic component being a frequency component of a harmonic; and an inverse orthogonal transformation unit that inversely orthogonally transforms the frequency data subjected to the filtering.

As described above, in the heart sound denoising apparatus reflecting one aspect of the present invention, the noise removal unit orthogonally transforms the resampled heart sound signal, extracts at least a fundamental frequency component, which is a frequency component of a fundamental wave, and a harmonic component, which is a frequency component of a harmonic, from frequency data after the orthogonal transformation, and inversely orthogonally transforms the extracted frequency data, so that it is possible to extract only the fundamental frequency component and the harmonic component of the heart sound signal synchronized with the pulsation signal and to remove only unwanted noise with certainty.

The heart sound denoising apparatus reflecting one aspect of the present invention is further provided with a pulse wave signal acquisition unit that acquires a pulse wave signal indicating a change in a flow of blood associated with a pulsation, wherein the pulsation signal acquisition unit acquires an electrocardiac signal indicating an electrical change associated with the pulsation, the first resampling unit resamples the acquired pulse wave signal in a same manner as the heart sound signal, and the noise removal unit removes a noise of the heart sound signal resampled by the first resampling unit, by an adaptive filter, using the pulse wave signal resampled by the first resampling unit as a reference signal.

As described above, in the heart sound denoising apparatus reflecting one aspect of the present invention, an electrocardiac signal indicating an electrical change associated with a pulsation and a pulse wave signal indicating a change in a flow of blood associated with the pulsation are acquired; the first resampling unit resamples the acquired pulse wave signal in a same manner as the heart sound signal; and the noise removal unit removes a noise of the heart sound signal resampled by the first resampling unit, by an adaptive filter, using the pulse wave signal resampled by the first resampling unit as a reference signal, so that it is possible to extract a clear heart sound signal from which the noise has been removed in synchronization with the pulse wave signal, by using, as the reference signal, the pulse wave signal which is synchronized more closely with the heart sound signal than the electrocardiac signal the pulse.

A heart sound denoising apparatus reflecting one aspect of the present invention is provided with: a heart sound signal acquisition unit that acquires a heart sound signal collected by a sound collector; a pulsation signal acquisition unit that acquires a pulsation signal of a living body; and a noise removal unit that removes a noise of the heart sound signal by an adaptive filter using the pulsation signal as a reference signal.

As described above, in the heart sound denoising apparatus reflecting one aspect of the present invention, a heart sound signal is acquired; a pulsation signal of a living body is acquired; and a noise of the heart sound signal is removed by an adaptive filter using the pulsation signal as a reference signal, so that it is possible to extract only a heart sound component synchronized with the pulsation signal, from the heart sound signal which is made up of a majority of external noise components and to remove most of the noise.

### BRIEF DESCRIPTION OF DRAWINGS

The advantages and features provided by one or more embodiments of the invention will become more fully understood from the detailed description given hereinbelow and the appended drawings which are given by way of illustration only, and thus are not intended as a definition of the limits of the present invention.

FIG. 1 is a hardware configuration diagram of a heart sound denoising apparatus according to a first embodiment;

FIG. 2 is a functional block diagram of the heart sound denoising apparatus according to the first embodiment;

FIG. 3A is a diagram showing an example of an electrocardiac signal;

FIG. 3B is a diagram showing an example of a heart sound signal;

FIG. 4A is a diagram showing a heart sound signal before resampling;

FIG. 4B is a diagram showing a heart sound signal after the resampling;

FIG. 5A to FIG. 5C are diagrams showing a process of performing band division by the modified discrete cosine transform (MDCT);

FIG. 6 is a diagram showing an example of a heart sound signal as a processing result of the heart sound denoising apparatus according to the first embodiment;

FIG. 7 is a flowchart showing an operation of the heart sound denoising apparatus according to the first embodiment;

FIG. 8 is a functional block diagram of a heart sound denoising apparatus according to a second embodiment;

FIG. 9 is a block diagram showing a processing of an adaptive filter;

FIG. 10 is a flowchart showing an operation of the heart sound denoising apparatus according to the second embodiment;

FIG. 11 is a functional block diagram of a heart sound denoising apparatus according to a third embodiment; and

FIG. 12 is a flowchart showing an operation of the heart sound denoising apparatus according to the third embodiment.

### DETAILED DESCRIPTION OF EMBODIMENTS

Hereinafter, one or more embodiments of the present invention will be described below with reference to the drawings. The same reference numerals are given to the same elements throughout the embodiments. However, the scope of the invention is not limited to the disclosed embodiments.

### First Embodiment

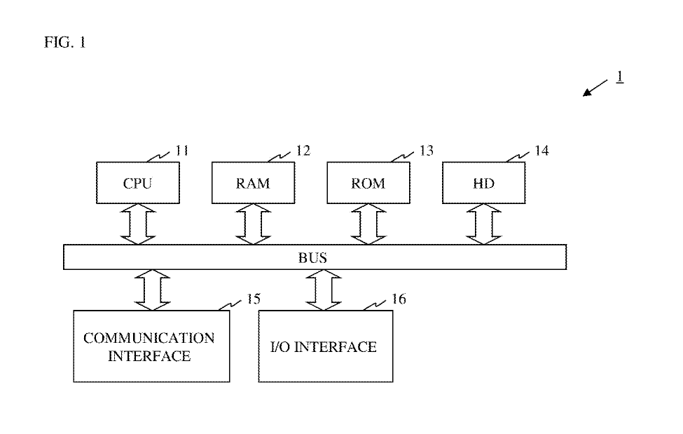

A heart sound denoising apparatus according to the present embodiment will be described with reference to FIG. 1 to FIG. 7. FIG. 1 is a hardware configuration diagram of a heart sound denoising apparatus according to the present embodiment. The heart sound denoising apparatus 1 is provided with a central processing unit (CPU) 11, a random access memory (RAM) 12, a read-only memory (ROM) 13, a hard disk (HD) 14, a communication interface 15, and an input/output (I/O) interface 16. An operating system and various programs are stored in the ROM 13 and the HD 14, read out to the RAM 12 as necessary, and each program is executed by the CPU 11.

The communication interface 15 is an interface for performing communication between other devices. The I/O interface 16 is an interface for accepting inputs from input devices such as a keyboard and a mouse, and for outputting data to a printer, a monitor, or the like. This I/O interface 16 can connect drives compatible with removable disks such as a magneto-optical disk, a floppy disk, a CD-R, a DVD-R and the like. It also functions as an interface compatible with storage media such as a USB memory, an SD (HC) card, a micro SD and the like. Each processing unit is connected via a bus and exchanges information.

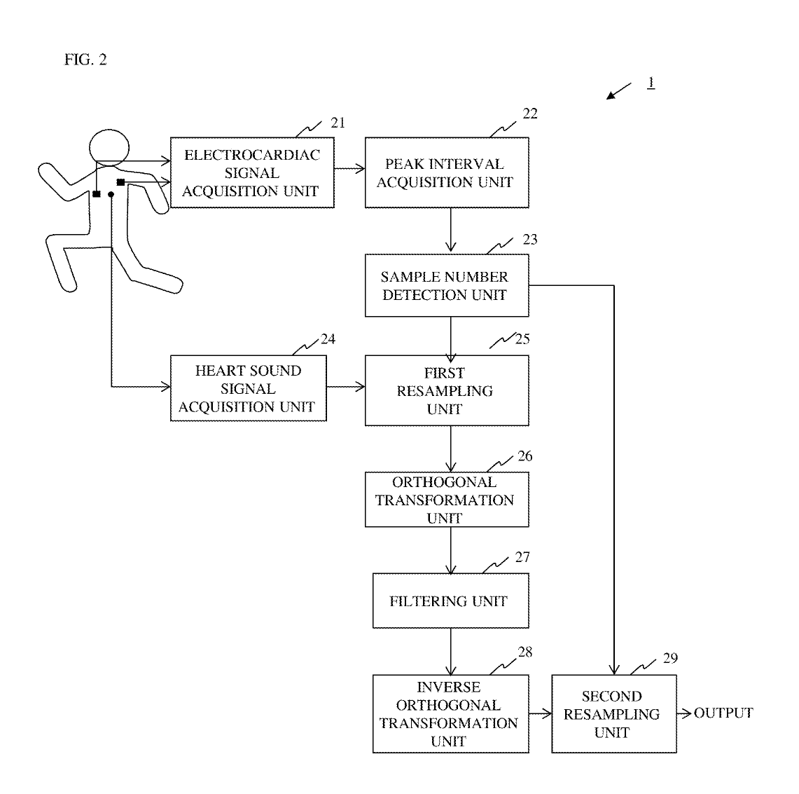

FIG. 2 is a functional block diagram of the heart sound denoising apparatus according to the present embodiment. A heart sound denoising apparatus 1 is provided with: an electrocardiac signal acquisition unit (also referred to as an electrocardiac signal acquirer) 21 that acquires an electrocardiac signal which is an electrical change associated with a pulsation obtained from an electrocardiac signal electrode attached to a person to be measured; a peak interval acquisition unit (also referred to as a peak interval acquirer) 22 that detects peaks of the acquired electrocardiac signal and acquires a peak interval rr for each adjacent pair of the peaks; a sample number detection unit (also referred to as a sample number detector) 23 that detects the number of samples at each peak interval (hereinafter referred to as the number of detected samples); a heart sound signal acquisition unit (also referred to as a heart sound signal acquirer) 24 that acquires a heart sound signal obtained from a sound collector attached to the person to be measured; a first resampling unit (also referred to as a first resampler) 25 that applies, to the heart sound signal, a processing to resample the number of samples of the electrocardiac signal obtained by the sample number detection unit 23 to the predetermined number of reference samples (preferably 2ⁿ); an orthogonal transformation unit (also referred to as an orthogonal transformer) 26 that orthogonally transforming the resampled heart sound signal; a filtering unit (also referred to as a filter) 27 that extracts only a fundamental frequency component and a harmonic component(s) from frequency data obtained by the orthogonal transformation; an inverse orthogonal transformation unit (also referred to as an inverse orthogonal transformer) 28 that inversely orthogonally transforms the filtered frequency data to generate a heart sound signal which has been resampled with the number of reference samples and from which a noise has been removed; and a second resampling unit (also referred to as a second resampler) 29 that resamples the inversely orthogonally transformed heart sound signal by the number of detected samples to output a heart sound signal at the time of detection from which the noise has been removed.

The peak interval acquisition unit 22 and the sample number detection unit 23 detect the peaks in the acquired electrocardiac acquire the interval between each pair of the detected peaks, and acquire the number of samples between each pair of the detected peaks as the number of detected samples. In addition, the peak detection can be carried out by using a generally known peak hold circuit or the like. For example, the technique disclosed in Patent Document 2 can be used for the peak detection.

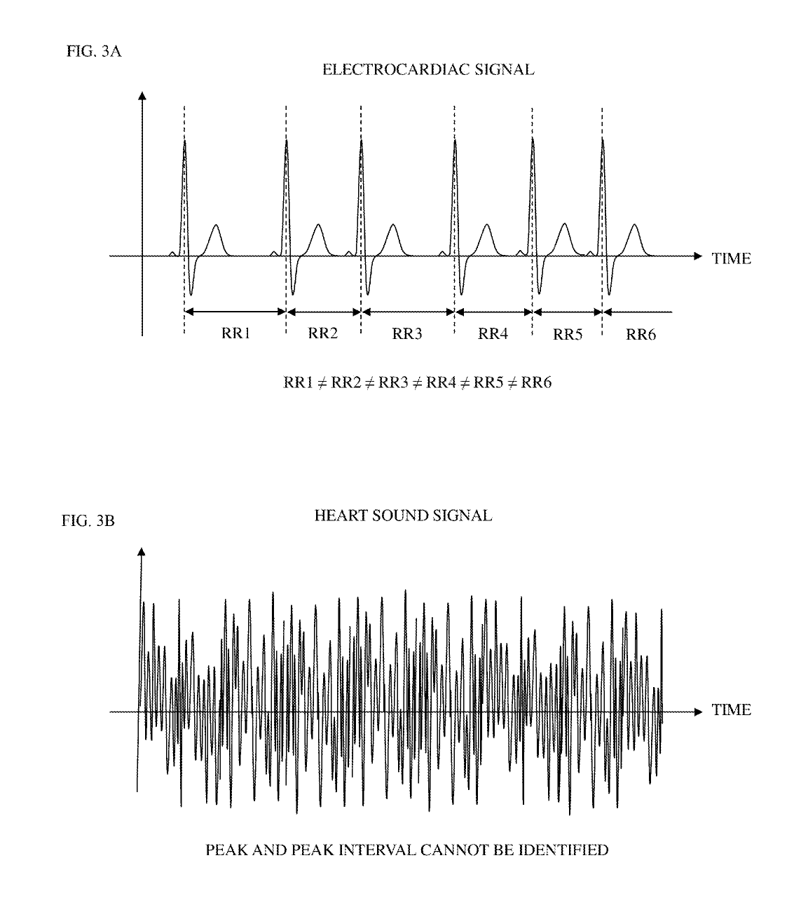

The first resampling unit 25 utilizes the feature that the peak interval of the electrocardiac signal and the heart sound are different in phase but synchronized, to resample the heart sound signal with the number of reference samples by using the number of detected samples of the peak interval of the acquired electrocardiac signal. FIG. 3A and FIG. 3B are diagrams showing an example of an electrocardiac signal and a heart sound signal. FIG. 3A shows the electrocardiac signal and FIG. 3B shows the heart sound signal. As shown in FIG. 3A, it is possible to acquire an electrocardiac signal with higher accuracy even in the case of intense motion or vibration such as during exercise, for example. However, the peak interval RR is not constant and is constantly changing, particularly in the case of during exercise. For example, a heart rate gradually increases at the beginning of an exercise, there appears a vertical variation during exercise, and the heart rate gradually decreases at the end of the exercise. In contrast, as shown in FIG. 3B, a heart sound signal is too noisy as it is impossible to distinguish it from a noise, particularly in the case of during exercise, and it is impossible to detect the heart sound signal. Therefore, in the present embodiment, a noise of the heart sound signal is eliminated based on the peak interval of the electrocardiac signal by utilizing the above-mentioned feature that the electrocardiac signal and the heart sound signal are synchronized with each other.

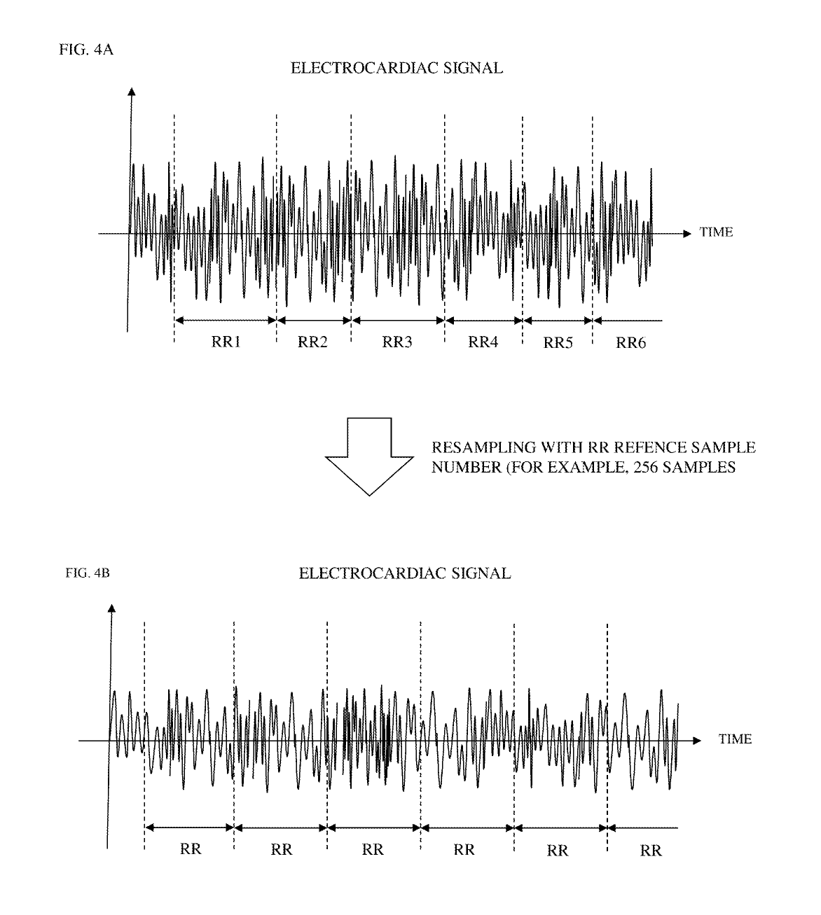

FIG. 4A and FIG. 4B are diagrams showing a signal in a case where the resampling process is performed on a heart sound signal by the first resampling unit 25. Based on the number of detected samples corresponding to the peak intervals RR1, RR2, RR3, . . . of the electrocardiac signal shown in FIG. 3A, the heart sound signal in FIG. 3B is resampled to the interval RR of the preset number of reference samples. That is, as shown in FIG. 4A and FIG. 4B, there is always a heart sound peak corresponding to RR1 within the RR1 of the heart sound signal, so that a period of RR1 in the heart sound signal is resampled to RR. Similarly, periods of RR2 and RR3 in the heart sound signal are resampled to RR. In this manner, the entire heart sound signal is resampled with the number of reference samples in RR.

When resampled to the number of reference samples, the orthogonal transformation unit 26 performs an orthogonal transformation process to convert the resampled waveform data into frequency data. As a method of the orthogonal transformation process, the discrete cosine transform (DCT), the modified DCT (MDCT), the lapped orthogonal transform (LOT), the Walsh-Hadamard transform (WHT) or the like can be used, for example. Here, it is assumed that an DCT conversion is performed.

The DCT transform functions as a plurality of filter banks, and there are N band-pass filters (BPFs). However, an orthogonal transformation such as DCT is a decimation processing, so that signals divided into bands become DC signals. That is, as described above, if such a process to normalize signals in a time axis direction is performed by the resampling process, a waveform having a harmonic of an integral multiple such as a periodic signal is necessarily converted into a DC component. In other words, the noise component does not have a harmonic structure and thus it is an AC component, so that it is possible to remove the noise component by extracting only the DC component with the low-pass filter (LPF) by the processing of the filtering unit 27.

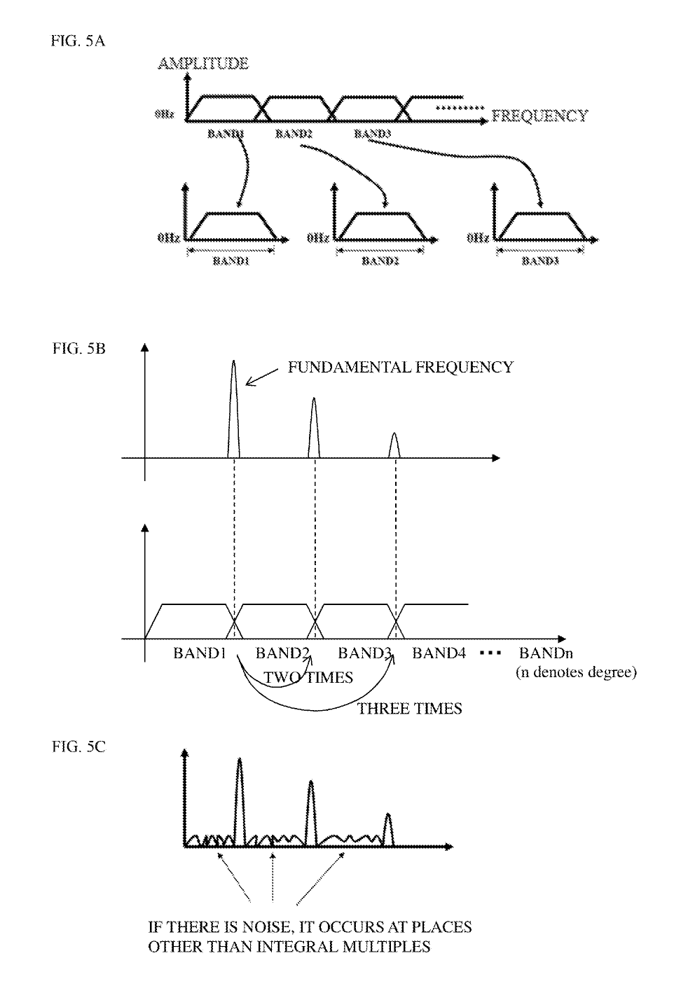

In addition, when the band division by MDCT is performed, it is divided into the same degree n as the number of reference samples (2ⁿ). Unlike ordinary BPF, the processing by the orthogonal transformation results in a width ranging from DC (0 Hz) to the BAND band as shown in FIG. 5A for each frequency band after the orthogonal transformation. That is, as shown in FIG. 5B, a normalized biosignal in a fixed period has a frequency generated at an integral multiple of a fundamental frequency, and, by matching the frequency of the integral multiple and the hand frequency after the orthogonal transformation, a result after the transformation becomes only slow DC changes, and a noise is generated in a region other than an integer multiple as shown in FIG. 5C.

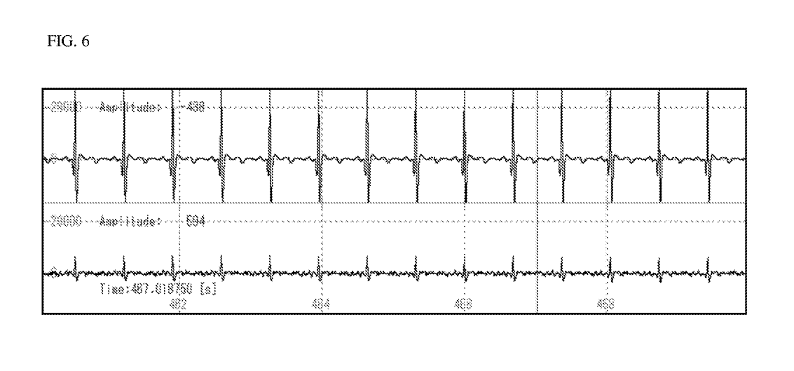

After the orthogonal transformation, the filtering unit 27 extracts the fundamental frequency component and the harmonic component for each frequency data after the transformation, and the inverse orthogonal transformation unit 28 performs the transform processing opposite to that of the orthogonal transformation unit 26. The second resampling unit 29 resamples the waveform data generated by the inverse transformation with the number of detected samples which is detected by the sample number detection unit 23, and returns the resampled data to the number of samples at the time of detection and outputs it. An example of the output result is shown in FIG. 6. The waveform shown at the upper part of FIG. 6 is a waveform of the electrocardiac signal and the waveform shown at the lower part of FIG. 6 is a heart sound signal from which a noise has been removed. As shown in FIG. 6, it is possible to detect a heart sound signal which is synchronized with an electrocardiac signal and from which a noise has been removed, by performing the processing according to the present embodiment.

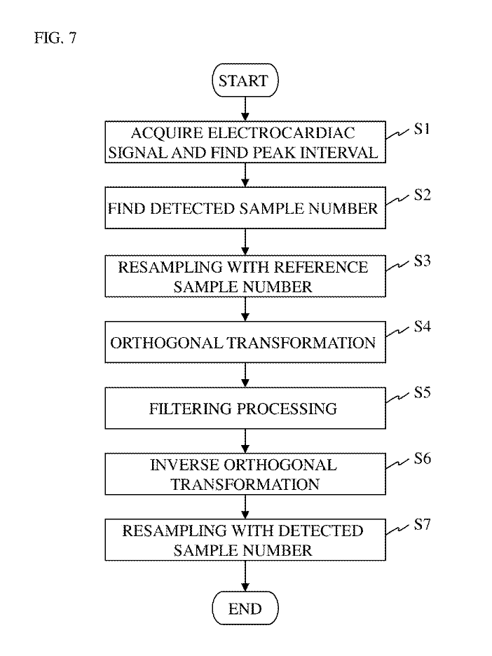

Next, the operation of the heart sound denoising apparatus according to the present embodiment will be described. FIG. 7 is a flowchart showing the operation of the heart sound denoising apparatus according to the present embodiment. First, the peak interval acquisition unit 22 detects the peaks in the electrocardiac signal acquired from the person to be measured by the electrocardiac signal acquisition unit 21, and obtains the peak interval for each adjacent pair of the peaks (S1). The sample number detection unit 23 obtains the number of samples in the obtained peak interval as the number of detected samples (S2). The first resampling unit 25 performs the resampling process on the heart sound signal acquired by the heart sound signal acquisition unit 24 by using the obtained number of detected samples and the preset number of reference samples (S3), Specifically, the heart sound signal in the period corresponding to the number of detected samples of the electrocardiac signal is resampled in the period of the number of reference samples.

In addition, the number of reference samples is desirably set to 2ⁿ or may be set based on an average value, a minimum number of samples or a maximum number of samples of the detected number of detected samples. It is particularly desirable to set it to be not less than the maximum value of the number of detected samples and the nearest 2ⁿ. By doing this, it is possible to minimize an error in interpolation processing while efficiently performing processing.

The orthogonal transformation unit 26 orthogonally transforms the heart sound signal resampled to the number of reference samples (S4). The filtering unit 27 filters the orthogonally-transformed frequency data to extract a fundamental frequency component and a harmonic component (S5). That is, a noise component other than the fundamental frequency component and the harmonic component is removed. The inverse orthogonal transformation unit 28 performs an inverse orthogonal transformation processing on the frequency data from which the noise component has been removed (S6). The second resampling unit 29 resamples the signal data obtained by the inverse orthogonal transformation with the number of detected samples obtained in S2 (S7). By the above series of steps, a new heart sound signal from which only the noise component has been removed from the original heart sound signal is outputted.

As described above, in the heart sound denoising apparatus according to the present embodiment, the peak interval of the electrocardiac signal is acquired; with respect to the heart sound signal detected at the same time as the electrocardiac signal, the heart sound signal corresponding to the acquired peak interval is resampled to a predetermined number of reference samples; and the noise component is removed from the resampled heart sound signal, so that it is possible to extract only the heart sound component according to the peak interval acquired from the electrocardiac signal, from the heart sound signal which is made up of a majority of external noise components (such as a worn sound of clothes during exercise, an environmental sound, and a wind sound, for example) and to remove most of the noise.

The removal of the noise component is performed by: orthogonally transforming the resampled heart sound signal; extracting at least the fundamental frequency component, which is the frequency component of the fundamental wave, and the harmonic component, which is the frequency component of the harmonic, from the frequency data after the orthogonal transformation; inversely orthogonally transforming the extracted frequency data; and resampling the heart sound signal to the number of samples at the peak interval (i.e., the number of detected samples) after the inverse orthogonal transformation, so that it is possible to extract only the fundamental frequency component and the harmonic component of the heart sound signal synchronized with the electrocardiac signal and to remove only unwanted noise with certainty.

In addition, the process of acquiring the electrocardiac signal as the pulsation signal and removing the noise of the heart sound signal based on the acquired electrocardiac signal has been described in the present embodiment. However, such a process of acquiring, as the pulsation signal, a pulse wave signal indicating a change in a flow of blood associated with the pulsation, and removing the noise of the heart sound signal by the above processing based on the pulse wave signal, may be used.

### Second Embodiment

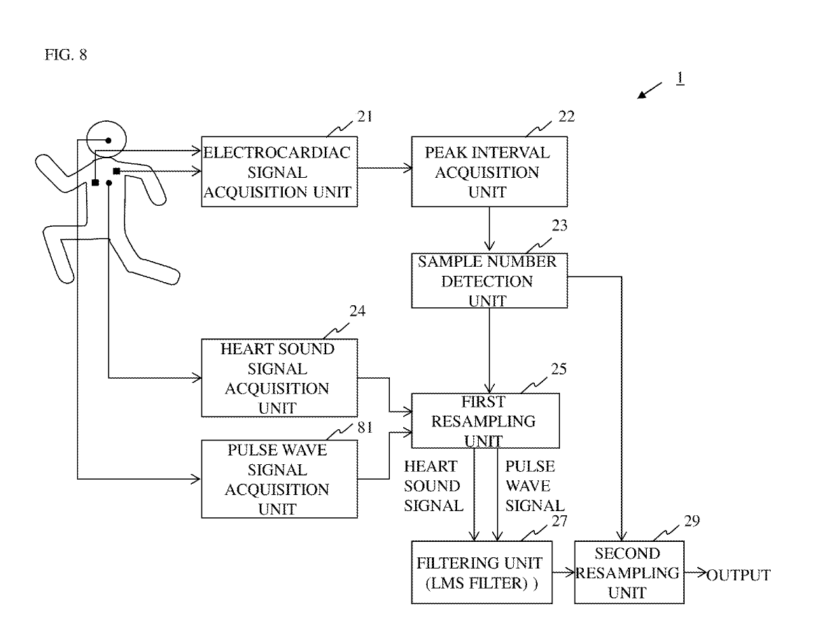

A heart sound denoising apparatus according to the present embodiment will be described with reference to FIG. 8 to FIG. 10. The heart sound denoising apparatus according to the present embodiment has made the processing of the heart sound denoising apparatus according to the first embodiment more accurate, and is configured to acquire a pulse wave signal of a blood, resample the pulse wave signal as well as the heart sound signal with the number of reference samples, and filter the resampled respective heart sound signal and pulse wave signal by an adaptive filter (for example, a least mean square (LMS) filter) to remove a noise. In addition, the description overlapping with the first embodiment will be omitted in the present embodiment.

FIG. 8 is a functional block diagram of the heart sound denoising apparatus according to the present embodiment. The heart sound denoising apparatus according to the present embodiment is provided with: an electrocardiac signal acquisition unit 21 that acquires an electrocardiac signal obtained from an electrocardiac signal electrode attached to a person to be measured; a peak interval acquisition unit 22 that detects peaks of the acquired electrocardiac signal and acquires a peak interval rr for each adjacent pair of the peaks; a sample number detection unit 23 that detects the number of samples in each peak interval (hereinafter referred to as the number of detected samples); a heart sound signal acquisition unit 24 that acquires a heart sound signal obtained from a sound collector attached to the person to be measured; a pulse wave signal acquisition unit (also referred to as a pulse wave signal acquirer) 81 that acquires a pulse wave signal of a blood from a pulse sensor (for example, an infrared sensor that measures the amount of hemoglobin in the blood) attached to an earlobe or the like; a first resampling unit 25 that applies, to the heart sound signal and the pulse wave signal, a processing to resample the number of samples of the electrocardiac signal obtained by the sample number detection unit 23 to the predetermined number of reference samples set in advance (preferably 2ⁿ); a filtering unit 27 that uses the resampled pulse wave signal as a reference signal and performs a filtering process on the resampled heart sound signal with an adaptive filter; and a second resampling unit 29 that resamples the filtered heart sound signal by the number of detected samples to output a heart sound signal at the time of detection from which a noise has been removed.

The pulse wave signal is more closely synchronized with the heart sound signal and it can be measured more accurately even during intense movement such as during exercise. This pulse wave signal is utilized based on the fact that it has one peak at the timing slightly delayed from the heart sound signal and between each pair of adjacent peaks of the electrocardiac signal. That is, as described above, the heart sound signal and the pulse wave signal are resampled with the number of reference samples based on the peak interval of the electrocardiac signal (the resampling method is the same as in the case of the first embodiment), and the adaptive filter is applied, using the pulse wave signal that is easy to detect with high accuracy among the respective signals and is closely related to the heart sound signal, as a reference signal.

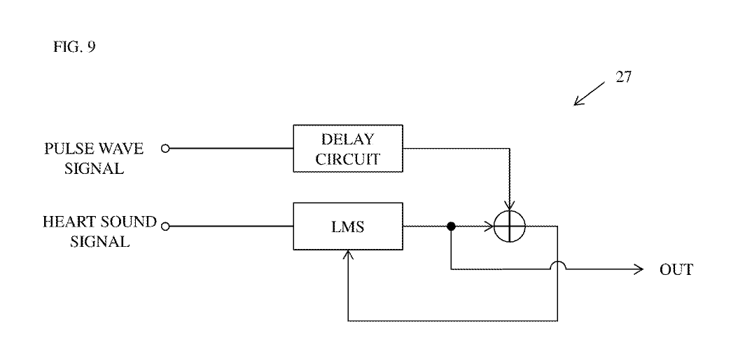

FIG. 9 is a block diagram showing a processing by the adaptive filter. As shown in FIG. 9, a heart sound signal including a noise is used as an input signal, and a pulse wave signal that can he acquired more accurately is used as a reference signal to apply to an adaptive filter. In the adaptive filter, the heart sound signal and the pulse wave signal are compared, and, by feeding back the error to an adaptive processor, a filter coefficient is controlled so that the root mean square of the error is minimized. As a result, a heart sound signal from which a noise has been removed is obtained as an output. Then, the second resampling unit 29 resamples the heart sound signal filtered with the number of detected samples, so that it is possible to output the heart sound signal at the time of detection from which the noise has been removed.

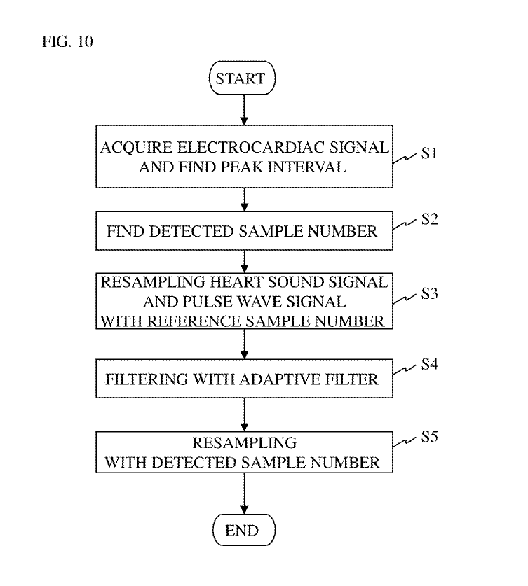

Next, the operation of the heart sound denoising apparatus according to the present embodiment will he described. FIG. 10 is a flowchart showing the operation of the heart sound denoising apparatus according to the present embodiment. First, the peak interval acquisition unit 22 detects the peaks in the electrocardiac signal acquired from the person to be measured by the electrocardiac signal acquisition unit 21, and obtains the peak interval for each adjacent pair of the peaks (S1). The sample number detection unit 23 obtains the number of samples in the obtained peak interval as the number of detected samples (S2). The first resampling unit 25 uses the obtained number of detected samples and the preset number of reference samples to perform a resampling process on the heart sound signal acquired by the heart sound signal acquisition unit 24 and the pulse wave signal acquired by the pulse wave signal acquisition unit 81 (S3). In addition, the resampling method is the same as in the first embodiment as described above, and the resampling process performed on the heart sound signal and the resampling process performed on the pulse wave signal are same.

The filtering unit 27 performs a filtering process with the adaptive filter using the pulse wave signal as a reference signal, for the heart sound signal and the pulse wave signal which has been resampled to the number of reference samples (S4). A noise of the heart sound signal is removed by this filtering process. Then, the second resampling unit 29 resamples the filtered signal data by the number of detected samples obtained in S2 (S5). By the above series of steps, a new heart sound signal from which only the noise component has been removed from the original heart sound signal is outputted.

As described above, in the heart sound denoising apparatus according to the present embodiment, the pulse wave signal of the blood is acquired, the acquired pulse wave signal is resampled in the same manner as the heart sound signal, and the resampled pulse wave signal is used as a reference signal to remove the noise of the heart sound signal by the adaptive filter, so that it is possible to extract a clear heart sound signal which synchronizes with the pulse wave signal and from which the noise has been removed, by using the pulse wave signal more closely synchronized with the heart sound signal than the electrocardiac signal as the reference signal.

### Third Embodiment

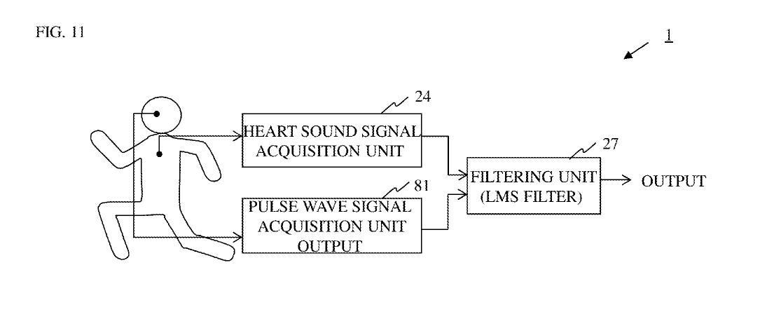

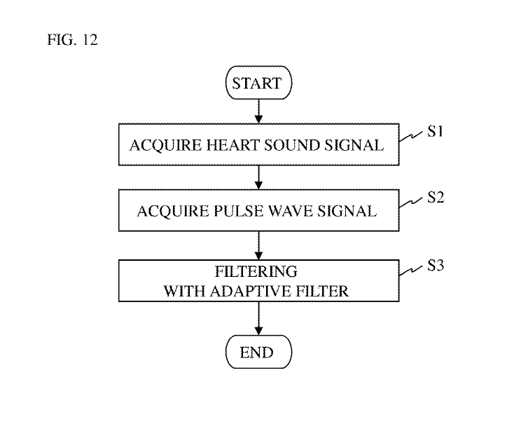

A heart sound denoising apparatus according to the present embodiment will be described with reference to FIG. 11 and FIG. 12. The heart sound denoising apparatus according to the present embodiment uses apulse wave signal of a blood as a reference signal and filters a heart sound signal with an adaptive filter (for example, an LMS filter) to remove a noise. In addition, the description overlapping with the first and second embodiments will be omitted in the present embodiment.

FIG. 11 is a functional block diagram of the heart sound denoising apparatus according to the present embodiment. The heart sound denoising apparatus according to the present embodiment is provided with: a heart sound signal acquisition unit 24 that acquires a heart sound signal obtained from a sound collector attached to a person to be measured; a pulse wave signal acquisition unit 81 that acquires a pulse wave signal of a blood from a pulse sensor (for example, an infrared sensor that measures the amount of hemoglobin in the blood) attached to an earlobe or the like; and a filtering unit 27 that uses the pulse wave signal acquired by the pulse wave signal acquisition unit 81 as a reference signal and performs a filtering process on the heart sound signal acquired by the heart sound signal acquisition unit 24 to output a result.

As described in the second embodiment, the pulse wave signal is closely synchronized with the heart sound signal, and it can be measured more accurately even during intense movement such as during exercise. This pulse wave signal is utilized based on the fact that it has peaks at the timing slightly delayed from the heart sound signal. That is, it is possible to remove a noise of the heart sound signal and output a result thereof by applying, to the adaptive filter, the pulse wave signal, which is easy to detect with higher accuracy and is closely related to the heart sound signal, as a reference signal. in addition, the processing of the adaptive filter is the same as that of FIG. 9 as described above.

Next, the operation of the heart sound denoising apparatus according to the present embodiment will be described. FIG. 12 is a flowchart showing the operation of the heart sound denoising apparatus according to the present embodiment. First, the heart sound signal acquisition unit 24 acquires a heart sound signal (S1). At the same time, the pulse wave signal acquisition unit 81 acquires a pulse wave signal (S2). The filtering unit 27 performs a filtering process on the acquired heart sound signal and pulse wave signal with the adaptive filter using the pulse wave signal as a reference signal (S3). By this filtering process, a new heart sound signal from which a noise of the heart sound signal has been removed is output.

As described above, in the heart sound denoising apparatus according to the present embodiment, the heart sound signal is acquired, the pulse wave signal of the blood is acquired, and the noise of the heart sound signal is removed by the adaptive filter using the pulse wave signal as the reference signal, so that it is possible to extract only the heart sound component synchronized with the pulse wave signal, from the heart sound signal which is made up of a majority of external noise components and to remove most of the noise, with a simple device configuration.

In addition, the process of acquiring the pulse wave signal of the blood from the pulse sensor attached to the earlobe or the like and using the pulse wave signal as the reference signal has been described in the present embodiment. However, such a process of acquiring an electrocardiac signal and using it as a reference to remove a noise may be used, for example.

Although embodiments of the present invention have been described and illustrated in detail, it is clearly understood that the same is by way of illustration and example only and not limitation, the scope of the present invention should not be interpreted by terms of the appended claims.

What is claimed is:

1. A heart sound denoising apparatus comprising:
a heart sound signal acquisition unit that acquires a heart sound signal collected by a sound collector;
a pulsation signal acquisition unit that acquires a pulsation signal of a living body;
a peak interval acquisition unit that acquires a peak interval of the acquired pulsation signal;
a first resampling unit that resamples the heart sound signal corresponding to the acquired peak interval, to a predetermined number of samples, with respect to the heart sound signal detected at a same time as the acquired pulsation signal;
a noise removal unit that removes a noise component from the resampled heart sound signal; and
a second resampling unit that resamples the heart sound signal resampled by the first resampling unit and with the noise removed, to a number of samples of the peak interval.

2. The heart sound denoising apparatus according to claim 1, wherein the pulsation signal is an electrocardiac signal indicating an electrical change associated with a pulsation, or a pulse wave signal indicating a change in a flow of blood associated with the pulsation.

3. The heart sound denoising apparatus according to claim 1, wherein the noise removal unit comprises:
an orthogonal transformation unit that orthogonally transforms the heart sound signal resampled by the first resampling unit;
a filtering unit that extracts at least a fundamental frequency component and a harmonic component from frequency data after the orthogonal transformation, the fundamental frequency component being a frequency component of a fundamental wave, the harmonic component being a frequency component of a harmonic; and
an inverse orthogonal transformation unit that inversely orthogonally transforms the frequency data subjected to the filtering.

4. The heart sound denoising apparatus according to claim 1, further comprising a pulse wave signal acquisition unit that acquires a pulse wave signal indicating a change in a flow of blood associated with a pulsation, wherein
the pulsation signal acquisition unit acquires an electrocardiac signal indicating an electrical change associated with the pulsation,
the first resampling unit resamples the acquired pulse wave signal in a same manner as the heart sound signal, and
the noise removal unit removes a noise of the heart sound signal resampled by the first resampling unit, by an adaptive filter, using the pulse wave signal resampled by the first resampling unit as a reference signal.

5. A heart sound denoising apparatus comprising:
a heart sound signal acquisition unit that acquires a heart sound signal collected by a sound collector;
a pulsation signal acquisition unit that acquires a pulsation signal of a living body; and
a noise removal unit that removes a noise of the heart sound signal by an adaptive filter using the pulsation signal as a reference signal.

6. A heart sound denoising method performedby a computer, the method comprising:
acquiring a heart sound signal collected by a sound collector;
acquiring a pulsation signal of a living body;
acquiring a peak interval of the acquired pulsation signal;
first-resampling the heart sound signal corresponding to the acquired peak interval, to a predetermined number of samples, with respect to the heart sound signal detected at a same time as the acquired pulsation signal;
removing a noise component from the resampled heart sound signal; and
second-resampling the heart sound signal first-resampled and with the noise removed, to a number of samples of the peak interval.

7. A program product that is stored in a non-transitory computer-readable recording medium executed by a computer, comprising the steps according to claim 6.

8. A computer medium embodying a computer program product according to claim 7.
